(19) United States
(12) Patent Application Publication
(10) Pub. No.: US 2022/0151868 A1
(43) Pub. Date: May 19, 2022

(54) Debris Removing Device And Method

(71) Applicant: James Rynerson, Franklin, TN (US)

(72) Inventor: James Rynerson, Franklin, TN (US)

(21) Appl. No.: 17/424,314

(22) PCT Filed: Jan. 27, 2020
(86) PCT No.: PCT/US2020/015171

§ 371 Date: Jul. 20, 2021

Related U.S. Application Data
(60) Provisional application No. 62/798,345, filed on Jan. 29, 2019.

(51) Int. Cl. A61H 35/02

(52) U.S. Cl. CPC A61H 35/02; A61H 2205/024; A61H 2201/0207

(57) ABSTRACT

A device (10) for removing debris from tissue around an eye is disclosed. The device (10) includes an eyecup (12a) having an opening (20a) and a cavity (18a), wherein the opening (20a) is of a size and shape sufficient to cover the eye. The device also includes an ultrasonic generator (60a) coupled to the eyecup (12a) and is configured to apply ultrasonic energy to a fluid in the eyecup (12a) to remove debris from the tissue. A method of removing debris from tissue around an eye a subject is also disclosed. The method includes covering the subjects eye with an eyecup (12a) having a cavity (18a) and filling the cavity (18a) of the eyecup (12a) with a fluid to bathe the eyelid margin with the fluid. Applying ultrasonic energy to the fluid at a frequency and for a duration sufficient to disrupt debris around the eye.

### CROSS-REFERENCE TO RELATED APPLICATIONS

This application claims the benefit of priority to U.S. Provisional Patent Application Ser. No. 62/798,345, filed Jan. 29, 2019 (pending), the disclosure of which is incorporated by reference herein.

### TECHNICAL FIELD

The present invention relates to devices for removing debris such as a biofilm from the tissue of the eyelids or, more particularly, the margins of the eyelid.

### BACKGROUND

Debris, including microbial biofilms, on the eyelid margin results in dry eye syndrome and blepharitis. In recent years, mechanical devices that directly contact the eyelid margin have been developed to remove debris from this region. However, while safe and effective, directly contacting the eyelid margin with a mechanical device can be frightening to a subject and if not done with great care, can result in discomfort and even damage to the delicate tissues of the eyelid margin. Methods and devices capable of disrupting and removing debris from the eyelid margin without mechanically contacting the eyelid margin are needed.

### SUMMARY

To these and other ends, a device (10) for removing debris from tissue of and around an eye including an eyelid margin is disclosed. The device (10) includes a first eyecup (12a) having an opening (20a) and a cavity (18a), wherein the opening (20a) is of a size and shape sufficient to cover the eye. The device further includes a first ultrasonic generator (60a) coupled to the first eyecup (12a) and configured to apply ultrasonic energy to a fluid in the first eyecup (12a) to remove debris from the tissue. The device may further comprise a first valve (40a) operatively coupled to the first eyecup (12a) for introducing the fluid into the cavity (18a) of the first eyecup (12a). The device may also include a second valve (40b) operatively coupled to the first eyecup (12a) for venting air from the cavity (18a) of the first eyecup (12a) when the first eyecup (12a) is being filled with the fluid.

In one embodiment, the device (10) may also include a second eyecup (12b) having an opening (20b) and a cavity (18b), wherein the opening (20b) is of a size and shape sufficient to cover a second eye. A second ultrasonic generator (60b) is coupled to the second eyecup (12b) and configured to apply ultrasonic energy to the fluid in the second eyecup (12b) to remove debris from the tissue. In this embodiment, the device (10) may further include a third valve (42a) operatively coupled to the second eyecup (12b) for introducing the fluid into the cavity (18b) of the second eyecup (12b) and a fourth valve (42b) operatively coupled to the second eyecup (12b) for venting air from the cavity (18b) of the second eyecup (12b) when the second eyecup (12b) is being filled with the fluid.

The device of any embodiment may include a sensor (80a) for sensing a temperature of the fluid in the first eyecup (12a). In these embodiments, the device (10) may also include a thermal generator (82a) operatively coupled to the first eyecup (12a) and configured to generate heat to increase the temperature of the fluid in the first eyecup (12a). In these embodiments, the device (10) may also include a controller (84) operatively coupled to the sensor (80a) and the thermal generator (82a), where the controller (84) is configured to receive from the sensor (80a) the temperature of the fluid in the first eyecup (12a) and activate the thermal generator (82a) to increase the temperature of the fluid in the first eyecup (12a) when the temperature drops below a desired temperature.

In one embodiment, the device (10) includes an electrode (66a) in the first eyecup (12a) configured to apply an electrical current to the liquid for a duration sufficient to disrupt and remove debris from the tissue in conjunction with the applied ultrasonic energy.

In one embodiment, a device (10) for removing debris from tissue of and around an eye including an eyelid margin includes first and second eyecups (12a, 12b), each eyecup (12a, 12b) having an opening (20a, 20b) and a cavity (18a, 18b), wherein each opening (20a, 20b) is of a size and shape sufficient to cover the eye. The device (10) includes first and second valves (40a, 42a), where the first valve (40a) is operatively coupled to the first eyecup (12a), and where the second valve (42a) is operatively coupled to the second eyecup (12b). Each of the first and second valves (40a, 42a) are configured to introduce a fluid into each eyecup (12a, 12b). The device (1) also includes third and fourth valves (40b, 42b), where the third valve (40b) is operatively coupled to the first eyecup (12a), and where the fourth valve (42b) is operatively coupled to the second eyecup (12b). Each of the third and fourth valves (40b, 42b) are configured to vent air from the cavity (18a, 18b) of each of the first and second eyecups (12a, 12b) when the first and second eyecups (12a, 12b) are being filled with the fluid. The device (10) further includes first and second ultrasonic generators (60a, 60b), where the first ultrasonic generator (60a) is operatively coupled to the first eyecup (12a), and where the second ultrasonic generator (60b) is operatively coupled to the second eyecup (12b). Each ultrasonic generator (60a, 60b) is configured to apply ultrasonic energy to a liquid in the first and second eyecups (12a, 12b) to remove debris from the tissue.

That embodiment may further include first and second sensors (80a, 80b) for sensing the temperature of the fluid in the first and second eyecups (12a, 12b), respectively and first and second thermal generators (82a, 82b) are operatively coupled to the first and second eyecups (12a, 12b), respectively. Each of the first and second thermal generators (82a, 82b) are configured to generate heat to increase the temperature of the fluid in the first and second eyecups (12a, 12b). The device (10) may also include a controller (84) operatively coupled to the first and second sensors (80a, 80b) and the first and second thermal generators (82a, 82b). The controller (84) is configured to receive from the first and second sensors (80a, 80b) the temperature of the fluid in the first and second eyecups (12a, 12b) and activate the first and second thermal generators (82a, 82b) to increase the temperature of the fluid in the first or second eyecups (12a, 12b) when the temperature drops below a desired temperature.

The invention also contemplates a system for removing debris tissue of and around an eye including an eyelid margin including the device (10) of any embodiment and a liquid. The liquid may be an isotonic aqueous liquid or a balanced salt solution.

The invention further contemplates a method of removing debris from tissue of and around an eye including an eyelid margin of a subject. The method includes covering the subject's eye with an eyecup (12a) having a cavity (18a) and forming a seal between a perimeter (24a) of the eyecup (12a) and the subject's eye; filling the cavity (18a) of the eyecup (12a) with a fluid to bathe the eyelid margin with the fluid; and applying ultrasonic energy to the fluid at a frequency and for a duration sufficient to disrupt debris on the eyelid margin. The method may also include maintaining the temperature of the liquid at a desired temperature. The method may also include applying an electrical current to the liquid for a duration sufficient to disrupt and remove debris from the eyelid margin in conjunction with the applied ultrasonic energy.

These and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a cross-sectional view of a tissue cleaning device according to one embodiment of the invention.

FIG. 2A is a schematic representation of the tissue cleaning device of FIG. 1 with fluid flowing in parallel to the two eye cups.

FIG. 2B is a schematic representation of the tissue cleaning device of FIG. 1 with fluid flowing in series to the two eye cups.

FIG. 3 is a schematic representation of a tissue cleaning device according to another embodiment of the invention.

### DETAILED DESCRIPTION

Disclosed are devices and methods for removing debris from tissue generally associated with an eye utilizing ultrasonic energy applied to a fluid bath in contact with the debris and eyelid margin. The disclosed devices and methods do not require mechanical contact with the tissue where mechanical contact is understood to mean physically contacting the tissue with a mechanical device to remove debris from this delicate tissue. As used herein, the tissue being treated is the eye and, more particularly, the eyelid and even more particularly, the margin of the eyelid from just external to the eyelashes to the eyelid that contacts the eyeball and that includes meibomian glands, eyelash follicles, glands of Moll, referred to herein as the eyelid margin.

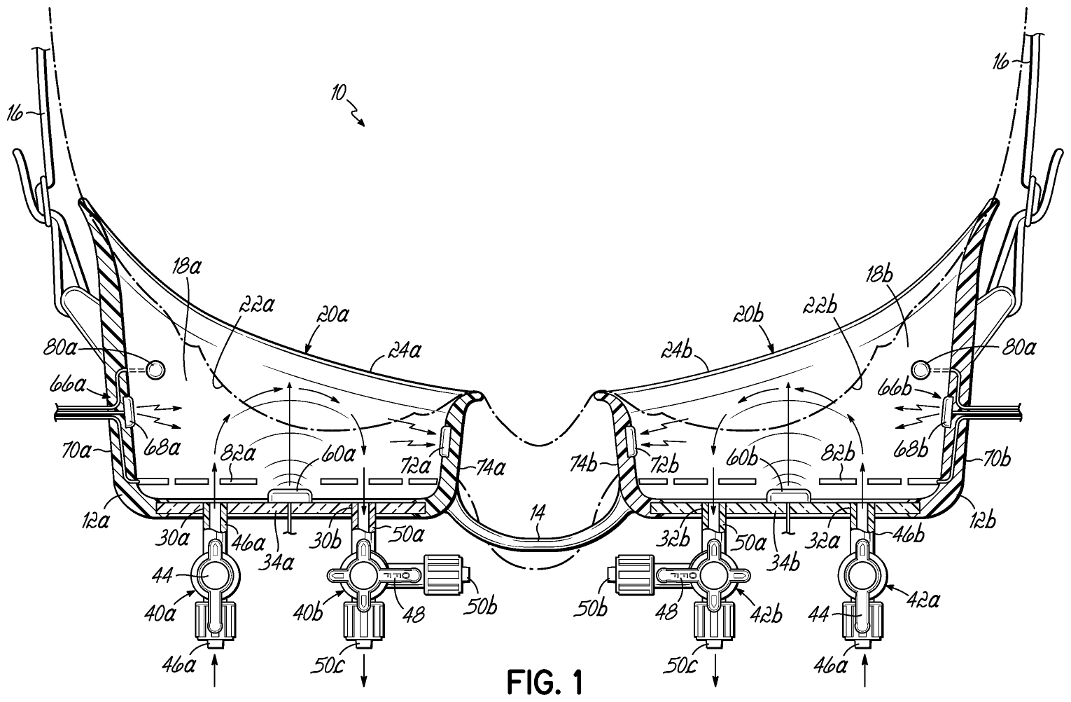

An exemplary embodiment of a debris removing device 10 constructed according to the principles of the invention is shown in FIG. 1. The device 10 includes right and left eyecups 12a, 12b which may be coupled together with a nose piece connecting member 14. The device 10 is configured to be secured to a subject's head via an adjustable strap 16. The nose piece connecting member 14 and strap 16 may be optional as the eyecups 12a, 12b may be held in place manually, with negative pressure applied to the eyecup 12a, 12b, with a wrap, or by other structures known in the art. The eyecups 12a, 12b have a cavity 18a, 18b with and opening 20a, 20b which allows access to the cavities 18a, 18b. The openings 20a, 20b are of a shape and size to permit the eyecups 12a, 12b to fit over the eyes 22a, 22b of a subject, including the eyelid margin from the eyelashes to the eyelid margin where the eyelid contacts the eyeball. A perimeter 24a, 24b of the openings 20a, 20b of the eyecups 12a, 12b may optionally also include a sealing structure, such as a pliable gasket formed from a rubber, silicone, or other elastomeric material capable of forming a seal between the subject's face and/or eye or eyelid and the cavities 18a, 18b of the eyecup 12a, 12b. The perimeter 24a, 24b may be shaped to generally conform to the curvature of the eye 22a, 22b.

Each eyecup 12a, 12b includes a pair of ports 30a, 30b, 32a, 32b passing through front panels 34a, 34b of the eyecups 12a, 12b. Valves 40a, 40b, 42a, 42b are operatively connected and are in fluid communication with the ports 30a, 30b, 32a, 32b, respectively. As illustrated in FIG. 1, valves 40a, 42a are two-way valves and valves 40b, 42b are three-way valves. Other valve configurations may be used as the circumstances warrant. The valves 40a, 42a include a valve handle 44 that may be turned to either allow fluid to flow in either direction through fluid ports 46a, 46b or block the fluid from flowing in either direction through fluid ports 46a, 46b. Similarly, the valves 40b, 42b include a handle 48 that may be turned to allow fluid to flow in either direction through at least two of three fluid ports 50a, 50b, 50c at any given time or to block fluid from flowing in either direction through one of the three fluid ports 50a, 50b, 50c.

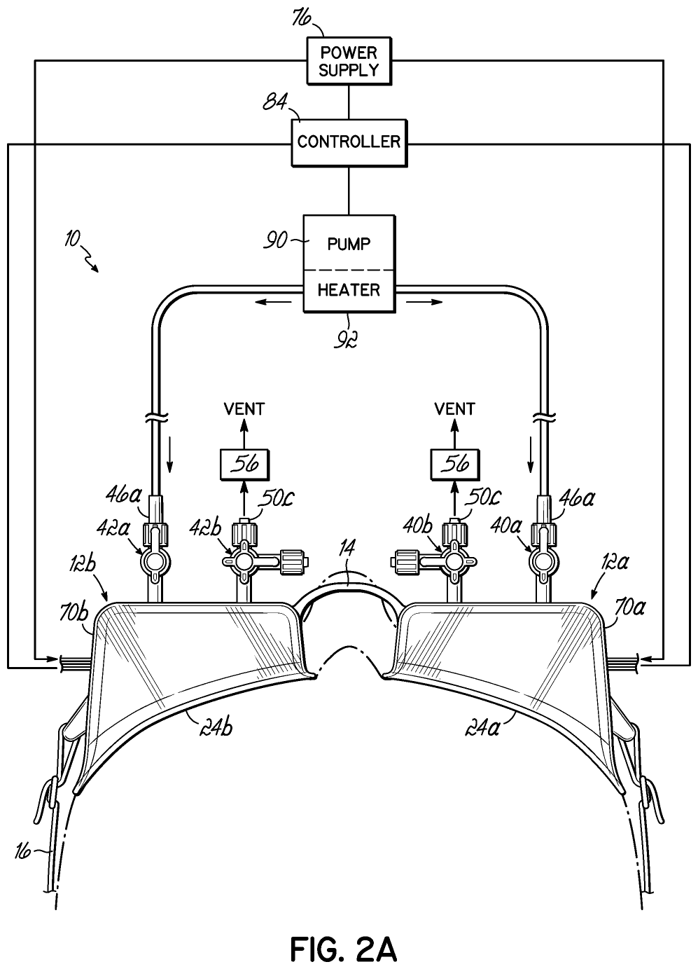

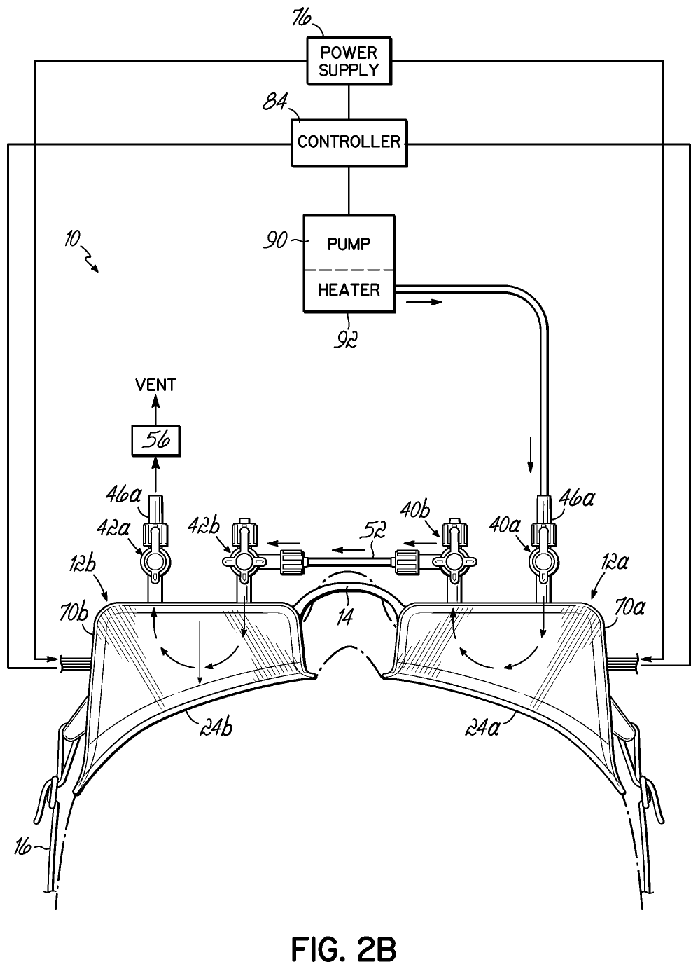

In one embodiment shown in FIG. 2A, a fluid enters the fluid port 46a of fluid valves 40a, 42a and exits fluid port 46b to fill the cavities 18a, 18b of the eyecups 12a, 12. As the fluid fills the cavities 18a, 18b, air from the cavities 18a, 18b is pushed out, i.e., vented, through ports 30b, 32b and is discharged out of fluid ports 50c of fluid valves 40b, 42b. In that configuration, fluid ports 50c of fluid valves 40b, 42b serve as air vents. When both cavities 18a, 18b are filled with fluid, the fluid valves 40a, 42b are closed so that no more fluid enters the cavities 18a, 18b. A small amount of fluid may be aspirated from the cavities 18a, 18b to provide a negative pressure in the cavities 18a, 18b, causing the eyecups 12a, 12b to suction to the area surrounding the subject's eyes 22a, 22b.

In one embodiment, a moisture sensor 56 may be operatively coupled to each of the fluid ports 50c and further operatively coupled to the controller 84. As the eyecups 12a, 12b begin to fill up with fluid and fluid flows out one of the fluid ports 50c, the moisture sensor 56 would sense the fluid and the controller 84 would shut off the pump 90 so no additional fluid will flow into the eyecup 12a, 12b that is full of fluid. At this point, the pump 90 may shut down completely or continue pumping fluid until the other eyecup 12a, 12b is full and the moisture sensor 56 senses fluid at the other fluid port 50c at which point the controller 84 would shut down the pump 90. Although FIG. 1 illustrates the ports 30a, 30b, 32a, 32b passing through front panels 34a, 34b of the eyecups 12a, 12b, those ports 30a, 30b, 32a, 32b may be positioned along the top surfaces of the eyecups 12a, 12b to ensure that the cavities 18a, 18b are filled completely with fluid before fluid begins to flow out of fluid ports 50c.

In another embodiment shown in FIG. 2B, fluid enters the fluid port 46a of valve 40a and exits fluid port 46b and fills cavity 18a of eyecup 12a. As the fluid fills the cavity 18a, air in cavity 18a is pushed out, i.e., vented, through port 30b, through fluid port 50b, through a connecting tube 52 coupled to and in fluid communication with fluid port 50b of valve 40b and fluid port 50b of valve 42b, and ultimately out of fluid port 46a of fluid valve 42a. In that configuration, fluid port 46a of fluid valve 42a serves as an air vent. As cavity 18a becomes full of fluid, the fluid begins to flow through the connecting tube 52 and into cavity 18b of eyecup 12b. When both cavities 18a, 18b are filled with fluid, the fluid valve 40a is closed so that no more fluid may enter the cavities 18a, 18b. A small amount of fluid may be aspirated from the cavities 18b to provide a negative pressure in the cavities 18a, 18b, causing the eyecups 12a, 12b to suction to the area surrounding the subject's eyes 22a, 22b. When the eyecups are held in place by the strap 16, it may not be necessary to aspirate the fluid to create the negative pressure in the cavities 18a, 18b. In one embodiment, the moisture sensor 56 may be operatively coupled to fluid port 46a and further operatively coupled to the controller 84. As eyecup 12b begins to fill up with fluid and fluid flows out of the fluid port 46a, the moisture sensor would sense the fluid and the controller 84 would shut off the pump 90 so no additional fluid will flow into eyecup 12a.

In one embodiment, each eyecup 12a, 12b includes an ultrasonic generator 60 (also known as an ultrasonic transducer) coupled to the front panels 34a, 34b such that when activated, ultrasonic energy is applied to and carried by the fluid in the cavities 18a, 18b of eyecups 12a, 12b. The ultrasonic energy carried by the fluid is then applied to the tissue generally associated with eyes 22a, 22b and debris on the tissue to disrupt the debris so that the debris may be removed from the tissue. The ultrasonic generator 60 may pass through the front panels 34a, 34b of the eyecups 12a, 12b so as to be in direct contact with the fluid, but not be in direct contact with the tissue. In an embodiment, the ultrasonic generator 60 may be waterproof or water resistant. An exemplary ultrasonic generator 60 is a piezo electric crystal. In an embodiment, the ultrasonic generator 60 may produce ultrasonic frequencies in the fluid at a frequency and for a duration sufficient to disrupt and remove the debris from the tissue. In an embodiment, the ultrasonic frequency may range between 20 kHz to 10 MHz. In an embodiment, the ultrasonic energy may be applied for a duration from a 10 seconds to 20 minutes or from 5 minutes to 20 minutes or from 10 minutes to 20 minutes, or from 5 minutes to 15 minutes, or from 10 minutes to 15 minutes, or from 5 minutes to 30 minutes, or from 10 minutes to 30 minutes, or from 15 minutes to 30 minutes.

In one embodiment, the eyecups 12a, 12b may also include electrodes 66a, 66b coupled to eyecups 12a, 12b to apply an electrical current to the fluid in the cavities 18a, 18b, which is in turn applied to the tissue and the debris on the tissue to disrupt the debris so that the debris may be removed from the tissue. The electrodes 66a, 66b have an anode 68a, 68b positioned in an outer edge 70a, 70b of the eyecups 12a, 12b from which the electric current enters the fluid and a cathode 72a, 72b positioned in an inner edge 74a, 74b of the eyecups 12a, 12b from which the electrical current leaves the fluid. The electrodes 66a, 66b may be powered by an external power supply 76 (FIGS. 2A, 2B).

The device 10 may also include sensors 80a, 80b, such as an electronic temperature sensor, for determining the temperature of the fluid in the cavities 18a, 18b. The device 10 may also include electric thermal generators 82a, 82b positioned within the cavities 18a, 18b to directly contact the fluid or embedded within the material forming the eyecups 12a, 12b such that they do not directly contact the fluid. The sensors 80a, 80b and the electric thermal generators 82a, 82b form part of a temperature regulation system that further includes a controller 84 (FIGS. 2A, 2B) operatively connected to the sensors 80a, 80b and the electric thermal generators 82a, 82b. An operator can set a desired temperature (or temperature range) for the fluid in the cavities 18a, 18b in the temperature regulation system and the sensors 80a, 80b may sense the fluid in the cavities 18a, 18b, report that to the controller 84, which may then signal the electric thermal generators 82a, 82b to heat the fluid in the cavities 18a, 18b should it fall below the desired temperature. In an embodiment, the desired temperature of the fluid in the cavities 18a, 18b is in the range of 37 degrees Celsius to 42 degrees Celsius. The controller 84 may also be operatively coupled to the power supply 76 so that the controller 84 may control the current supplied to the electrodes 66a, 66b. In one embodiment, a pump 90 (FIGS. 2A, 2B) with a heater 92 is used to initially heat the fluid before the fluid enters the cavities 18a, 18b. The pump 90 may further include a fluid reservoir (not shown) that may be filled with the fluid used to fill the eyecups 12a, 12b. Return tubes may be connected from the cavities 18a, 18b to the fluid reservoir so fluid in the cavities 18a, 18b may recirculated back to the pump and reheated to maintain the temperature of the fluid in the cavities 18a, 18b, at the desired temperature (or range). The controller 84 may be operatively coupled to the pump 90 and the heater 92 to control the recirculation and the re-heating of the fluid in the cavities 18a, 18b. The recirculation tube may be connected to the valves 40b, 42b or to a secondary port (not shown) in the eyecups 12a, 12b. The sensors 80a, 80b may be in communication with the controller 84 which may control how much heat the heater 92 applies to the recirculated fluid.

The fluid in the cavities 18a, 18b may be any fluid that the eye and the associated tissue may tolerate when ultrasonic energy is applied to the fluid. In an embodiment, the fluid is an aqueous liquid, and preferably an isotonic aqueous liquid, and even more preferably a buffered isotonic aqueous liquid. Exemplary liquids include hypertonic saline, balanced salt solutions (BSS) that have physiological pH, and an isotonic salt concentration. The liquid may further include an active agent such as a pharmacological agent that may assist in treating a medical condition. The active agent is provided in a dose sufficient to treat the medical condition. For example, the liquid may include an anti-inflammatory agent, an anti-fungal agent, an anti-bacterial agent, a surfactant, or combinations thereof. In embodiment, the liquid includes Restasis® (an ophthalmic emulsion of cyclosporine 0.05%), Xiidra (a lifitegrast ophthalmic solution 5%), or combinations thereof.

Debris may include a biofilm, an accumulation of dead cells such as skin cells, scurf, dirt, glandular plugs, makeup, chemical contaminants, and other material that is desired to be removed from the tissue. In an embodiment, the debris is a biofilm, such as a biofilm formed on the tissue by microorganisms.

Embodiments of the device may be used to treat a condition in which removal of debris from the tissue treats the condition or reduces the symptoms of the condition. The exemplary embodiment described below is configured to remove debris from the eye, and more particularly debris from the eyelid margin. Debris in the eyelid margin, especially biofilm on the eyelid margin and blockage of the meibomian glands can cause dry eye syndrome and blepharitis. Removing this debris with embodiments of the invention described herein may treat dry eye syndrome and blepharitis.

The embodiments of the debris removing device 10 described and illustrated herein have two eyecups 12a, 12b so that two eyes may be treated simultaneously. If only one eye requires treatment, or the treatment is to be applied to one eye at a time, a debris removing device may be constructed according to the principles of the invention, but with only one eyecup. Alternatively, the debris removing device 10 may be used to treat one eye where the fluid fills only one of the cavities 18a, 18b, instead of both as described above.

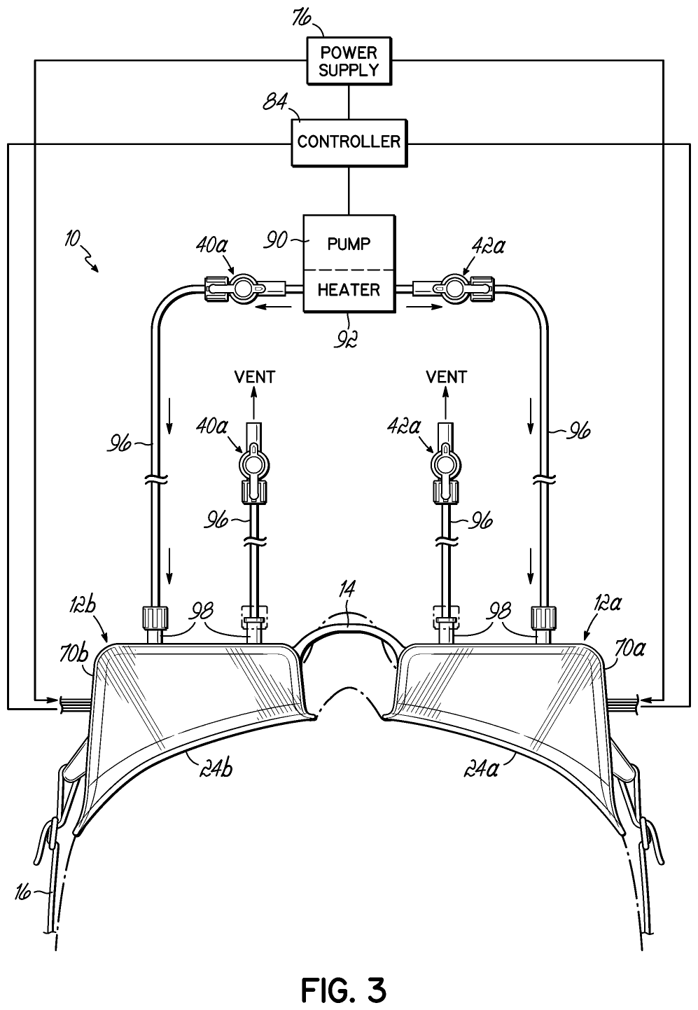

In FIGS. 1, 2A, and 2B, the fluid ports 46b, 50a on valves 40a, 40b, 42a, 42b are connected essentially directly to the ports 30a, 30b, 32a, 32b in close proximity to the eyecups 12a, 12b. In another embodiment shown in FIG. 3, valves, such as valves 40a, 42a, may be positioned remotely from the eyecups 12a, 12b, for instance closer to the pump 90 or the controller 84. In this embodiment, tubing 96 may be used to connect the valves 40a, 42a to connectors 98 which are coupled to the eyecups 12a, 12b. By locating the valves 40a, 42a remotely from the eyecups 12a, 12b, the device 10 may feel lighter to the subject wearing the device 10. Also, the device 10 may be more readily cleaned after being used by the subject. In addition, it may be easier for a doctor or technician to open and close the valves if they are positioned remotely from the eyecups 12a, 12b as illustrated in FIG. 3.

While the present invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. Further, it will be appreciated that the device may also include additional electronics, such as controllers and power supplies for the ultrasonic generator, optional electrodes, and optional temperature regulation system, that are not described herein as these electronics are well known in the art. The invention in its broader aspects is, therefore, not limited to the specific details, representative apparatus and method, and illustrative examples shown and described. Accordingly, departures may be made from such details without departing from the spirit or scope of the general inventive concept.

1. A device for removing debris from tissue of and around an eye including an eyelid margin, the device comprising:
a first eyecup having an opening and a cavity, wherein the opening is of a size and shape sufficient to cover the eye; and
a first ultrasonic generator coupled to the first eyecup and configured to apply ultrasonic energy to a fluid in the first eyecup to remove debris from the tissue.

2. The device of claim 1 further comprising a first valve operatively coupled to the first eyecup for introducing the fluid into the cavity of the first eyecup.

3. The device of claim 2 further comprising a second valve operatively coupled to the first eyecup for venting air from the cavity of the first eyecup when the first eyecup is being filled with the fluid.

4. The device of claim 1 further comprising a second eyecup having an opening and a cavity, wherein the opening is of a size and shape sufficient to cover a second eye; and
a second ultrasonic generator coupled to the second eyecup and configured to apply ultrasonic energy to the fluid in the second eyecup to remove debris from the tissue.

5. The device of claim 4 further comprising a third valve operatively coupled to the second eyecup for introducing the fluid into the cavity of the second eyecup.

6. The device of claim 5 further comprising a fourth valve operatively coupled to the second eyecup for venting air from the cavity of the second eyecup when the second eyecup is being filled with the fluid.

7. The device of claim 1 further comprising a sensor for sensing a temperature of the fluid in the first eyecup.

8. The device of claim 7 further comprising a thermal generator operatively coupled to the first eyecup and configured to generate heat so as to increase the temperature of the fluid in the first eyecup.

9. The device of claim 8 further comprising a controller operatively coupled to the sensor and the thermal generator, the controller configured to receive from the sensor the temperature of the fluid in the first eyecup and activate the thermal generator to increase the temperature of the fluid in the first eyecup when the temperature drops below a desired temperature.

10. The device of claim 1 further comprising an electrode in the first eyecup configured to apply an electrical current to the liquid for a duration sufficient to disrupt and remove debris from the tissue in conjunction with the applied ultrasonic energy.

11. A device for removing debris from tissue of and around an eye including an eyelid margin, the device comprising:
first and second eyecups, each eyecup having an opening and a cavity, wherein each opening is of a size and shape sufficient to cover the eye;
first and second valves, the first valve operatively coupled to the first eyecup, the second valve operatively coupled to the second eyecup, each of the first and second valves configured to introduce a fluid into each eyecup;
third and fourth valves, the third valve operatively coupled to the first eyecup, the fourth valve operatively coupled to the second eyecup each of the third and fourth valves configured to vent air from the cavity of each of the first and second eyecups when the first and second eyecups are being filled with the fluid; and
first and second ultrasonic generators, the first ultrasonic generator operatively coupled to the first eyecup, the second ultrasonic generator operatively coupled to the second eyecup, each ultrasonic generator configured to apply ultrasonic energy to a liquid in the first and second eyecups to remove debris from the tissue.

12. The device of claim 11 further comprising:
first and second sensor for sensing a temperature of the fluid in the first and second eyecups, respectively; and
first and second thermal generators operatively coupled to the first and second eyecups, respectively, and each of the first and second thermal generators configured to generate heat so as to increase the temperature of the fluid in the first and second eyecups.

13. The device of claim 12 further comprising a controller operatively coupled to the first and second sensors and the first and second thermal generators, the controller configured to receive from the first and second sensors the temperature of the fluid in the first and second eyecups and activate the first and second thermal generators to increase the temperature of the fluid in the first or second eyecups when the temperature drops below a desired temperature.

14. The device of claim 12 further comprising first and second electrodes in the first and second eyecups, respectively, the first and second electrodes configured to apply an electrical current to the liquid for a duration sufficient to disrupt and remove debris from the tissue in conjunction with the applied ultrasonic energy.

15. A system for removing debris tissue of and around an eye including an eyelid margin comprising:
the device of claim 1 and a liquid.

16. The system of claim 15, wherein the liquid is an isotonic aqueous liquid.

17. The system of claim 15, wherein the liquid is a balanced salt solution.

18. A method of removing debris from tissue of and around an eye including an eyelid margin of a subject comprising:
covering the subject's eye with an eyecup having a cavity and forming a seal between a perimeter of the eyecup and the subject's eye;
filling the cavity of the eyecup with a fluid to bathe the eyelid margin with the fluid; and
applying ultrasonic energy to the fluid at a frequency and for a duration sufficient to disrupt debris on the eyelid margin.

19. The method of claim 18 further comprising:
maintaining the temperature of the liquid at a desired temperature.

20. The method of claim 18 further comprising:
applying an electrical current to the liquid for a duration sufficient to disrupt and remove debris from the eyelid margin in conjunction with the applied ultrasonic energy.
